(19) United States
(12) Patent Application Publication
(10) Pub. No.: US 2010/0114031 A1
(43) Pub. Date: May 6, 2010

(54) INTRODUCER LOCALIZATION ASSEMBLIES

(76) Inventors: Inderjeet S. Jarial, Carmel, IN (US); Jason M. Butcher, Lafayette, IN (US); Jacob Flagle, New Palistine, IN (US); Jason Germann, Indianapolis, IN (US); Zachary R. Nicoson, Indianapolis, IN (US); William O. Hodge, Greenfield, IN (US); Samantha Perkins, Carmel, IN (US); Joseph L. Mark, Indianapolis, IN (US); Aaron P. Barr, Fishers, IN (US); Michael Hoffa, Brownsburg, IN (US)

Correspondence Address: Hologic Cytyc-Suros Division;c/o Rader, Fishman & Grauer, PLLC, 39533 Woodward Avenue, Suite 140, Bloomfield, MI, 48304, US

(21) Appl. No.: 12/612,422

(22) Filed: Nov. 4, 2009

Related U.S. Application Data
(60) Provisional application No. 61/111,604, filed on Nov. 5, 2008.
(60) Provisional application No. 61/122,172, filed on Dec. 12, 2008.

(51) Int. Cl. A61B 17/34

(52) U.S. Cl. 604/164.11

(57) ABSTRACT

An introducer assembly is disclosed. The introducer assembly comprises an introducer cannula, an introducer hub and an introducer stylet body. The introducer cannula is defined by a distal end and a proximal end and has a lumen extending therebetween. The introducer hub is operatively connected to the introducer cannula, and wherein the introducer hub is configured for selective connection with multiple medical devices. The introducer stylet body is defined by a proximal end and a distal end and is removably disposed within the introducer cannula. The introducer stylet body may further comprise an imagable feature having a predetermined size disposed on a distal end thereof.

### CROSS-REFERENCE TO RELATED APPLICATIONS

This application claims the benefit of U.S. Provisional Application Ser. Nos. 61/111,604 filed Nov. 5, 2008, and 61/122,172 filed Dec. 12, 2008, which applications are hereby incorporated by reference in their entirety.

### TECHNICAL FIELD

The present disclosure relates generally to introducer stylets and cannulas used for biopsy systems.

### BACKGROUND

Medical procedures have advanced to stages where less invasive or minimally invasive surgeries, diagnostic procedures and exploratory procedures have become desired and demanded by patients, physicians, and various medical industry administrators. To meet these demands, improved medical devices and instrumentation have been developed, such as cannulae or micro-cannulae, medical introducers, vacuum assisted biopsy apparatus, and other endoscopic related devices.

During certain biopsy procedures, an introducer stylet may be used to create a pathway to access the biopsy site. The introducer stylet is inserted into body tissue with a cannula mounted thereon. After the pathway is created, the introducer stylet is removed, but the cannula is left within the tissue pathway providing access to the biopsy area and further minimizing movement of the biopsy device adjacent a target lesion. To further locate the introducer stylet near an excision site, different systems have been used, such as open surgical procedures, mammography or magnetic resonance imaging (MRI). Ultrasonography may also be used to further assess target areas found and further used as a guidance method to better guide the introducer stylet adjacent suspect tissue.

In recent years, ultrasound-guided biopsy of breast lesions has been used for diagnostic purposes, partially replacing open surgical intervention. Ultrasound guidance is also often used for needle localization of some lesions prior to biopsy, as well as for cyst aspiration. Ultrasound guidance for such interventions, however, is difficult to learn and perform. One needs good hand-eye coordination and three-dimensional visualization skills to guide the biopsy needle to the target tissue area with the aid of ultrasound imagery. Devices have been developed that try to create a higher reflective property for viewing with the ultrasonic wave. However, these devices do not allow the operator to pinpoint exactly where the device sampling aperture is in relation to the lesion site nor do they afford the operator the ability to place the introducer stylet with the biopsy aperture up prior to inserting the biopsy device providing exact location to the tissue to be sampled.

Therefore, what is needed is an introducer stylet having an imagable feature capable of replicating the biopsy tool's aperture image signature, thus effectively identifying the location of the excision site while maintaining the ability to remove the introducer stylet. There is also a separate need for an introducer stylet having an orientation indicator with a selectively lockable and removable cannula introducer capable of receiving and orienting a biopsy device after removal of the introducer stylet. Finally, there is also a need for a selectively lockable cannula that may be selectively used with different types of medical devices.

### SUMMARY

In one exemplary arrangement, an introducer stylet is disclosed. The introducer stylet comprises an introducer stylet body defined by a proximal end and a distal end. The distal end further includes an imagable feature. The imagable feature may be configured with a predetermined size that generally corresponds to a portion of a medical device.

Also disclosed are various embodiments of introducer cannulas for use with various introducer stylets, including, but not limited to, the imagable introducer stylet also disclosed herein. The introducer cannulas disclosed also include various configurations of introducer hubs for attaching to various medical devices.

### BRIEF DESCRIPTION OF THE DRAWINGS

FIG. 1 is a perspective view of an introducer stylet;

FIG. 2 is a perspective view of the introducer stylet inserted into an introducer cannula and engaged in a locked position;

FIG. 3 is a perspective view of the introducer stylet and introducer cannula of FIG. 2 with the introducer stylet in an unlocked position;

FIG. 4A is a perspective view of the introducer cannula having a connecting hub.

FIG. 4B is a perspective view of the introducer cannula of FIG. 4A with syringe attached thereto;

FIG. 5A is a perspective view of an imagable feature on the introducer stylet representing the aperture of a biopsy device;

FIG. 5B is a perspective view of a turned diameter imagable feature on the introducer stylet representing the aperture of a biopsy device;

FIG. 5C is a perspective view of a machined faces imagable feature on the introducer stylet representing the aperture of a biopsy device; and

FIG. 6A is a side view of a spring loaded introducer stylet in a pre-fire position;

FIG. 6B is a side view of the spring loaded introducer stylet of FIG. 6A in a fired positioned.

FIG. 7A is a side perspective view of an alternative embodiment of a spring loaded introducer stylet in a pre-fire position.

FIG. 7B is a side perspective view of the spring loaded introducer stylet assembly of FIG. 7A in a fired position.

FIG. 8A is a partial cross-sectional and perspective view of the spring loaded introducer stylet assembly of FIG. 7A in the pre-fire position.

FIG. 8B is a partial cross-sectional and perspective view of the spring loaded introducer stylet assembly of FIG. 7A in the fired position.

FIG. 9A is an enlarged cross-sectional view of the spring loaded introducer stylet assembly of FIG. 8A in the pre-fire position.

FIG. 9B is an enlarged cross-sectional view of the spring loaded introducer stylet assembly of FIG. 8B in the fired position.

FIG. 10A is a perspective view of an introducer assembly with a multiple fitting introducer hub attached to a luer lock syringe.

FIG. 10B is a perspective view of the introducer assembly of FIG. 10A attached to a luer taper syringe.

FIG. 11A is a side view of the introducer assembly of FIG. 10A attached to a luer lock syringe.

FIG. 11B is a side view of the introducer assembly of FIG. 10B attached to a luer taper syringe.

FIG. 12A is a side cross-sectional view of the introducer assembly of FIG. 10A attached to a luer lock syringe.

FIG. 12B is a side cross-sectional view of the introducer assembly of FIG. 10B attached to a luer taper syringe.

### DETAILED DESCRIPTION

Referring now to the drawings, illustrative and exemplary embodiments of the present disclosure are shown in detail. Although the drawings represent some embodiments of the present disclosure, the drawings are not necessarily to scale and certain characteristics may be exaggerated to better illustrate and explain the present disclosure. Further, the embodiments set forth herein are not intended to be exhaustive or otherwise limit or restrict the disclosure to the precise forms and configurations disclosed in the following detailed description.

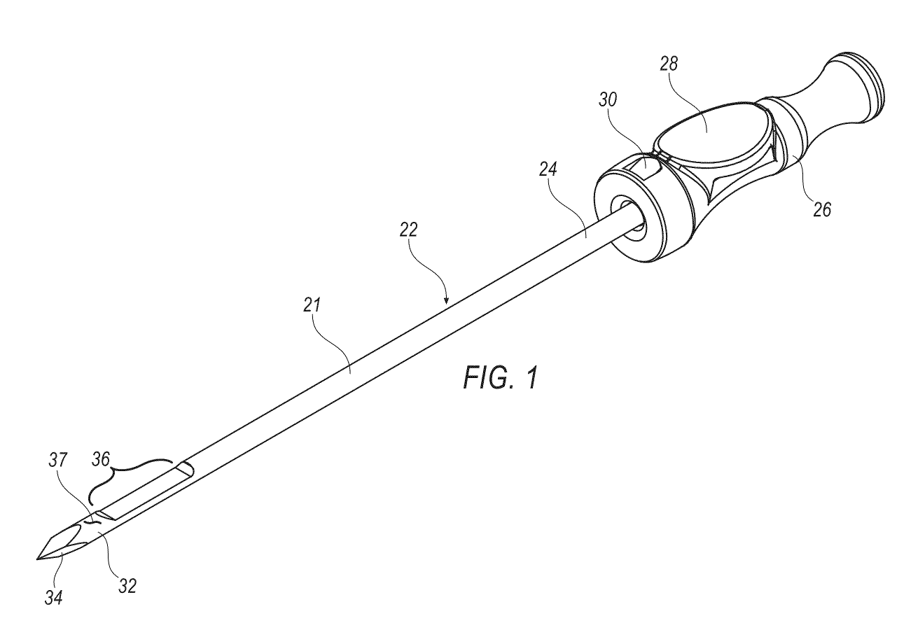

Turning now to the drawings, and in particular to FIG. 1, an exemplary introducer stylet body 22 of an introducer assembly is shown. In the arrangement of FIG. 1, introducer stylet body 22 is configured so as to be generally cylindrical and is defined by a proximal end 24 and a distal end 32. Introducer stylet body 22 may be configured to be generally solid. Distal end 32 includes a tissue piercing tip 34, such as, for example, a trocar tip, sized for introduction into a patient's body. In addition to a trocar tip, it will be appreciated that the piercing tip 34 may include other devices for piercing the patient's tissue, including without limitation, devices that use a laser or radio frequencies (RF) to pierce the tissue.

In the embodiment illustrated, introducer stylet body 22 extends from a handle 26 positioned opposite distal end 32. The handle 26 may include a latching tab interference portion 30, to be explained below in further detail. In one exemplary arrangement, handle 26 is fixed with respect to introducer stylet body 22 such that rotation of handle 26 causes introducer stylet body 22 to rotate as well.

In one exemplary arrangement, introducer stylet body 22 is provided with an imagable feature 36, disposed adjacent to distal end 32. In certain arrangements, imagable feature 36 (to be discussed in further detail below) may be, but is not required to be, configured so as to represent the size, as well as an orientation of an aperture of a biopsy device. In embodiments where an imagable feature 36 is provided that is configured to represent an orientation of an aperture of a biopsy device, an imagable feature orientation indicator 28 may be provided. In one specific, but exemplary arrangement, the imagable feature orientation indicator 28 may be configured as an ergonomic thumb pad. Imagable feature orientation indicator 28 provides a visual position of the imagable feature 36 (to be discussed in further detail below) on the introducer stylet body 22, relative to the handle 26 position. Alternatively, the imagable feature orientation indicator 28 may be configured by printing a mark on the handle (not shown) that allows the operator to orient the imagable feature 36 to a target tissue area prior to removal of the introducer stylet body 22 and insertion of a core biopsy device within an introducer cannula 42, to be explained in further detail below. An exemplary core biopsy apparatus may be seen by way of example in U.S. Pat. Nos. 6,638,235 and 6,744,824, which are commonly owned by the assignee of the present disclosure and are incorporated herein by reference in their entirety.

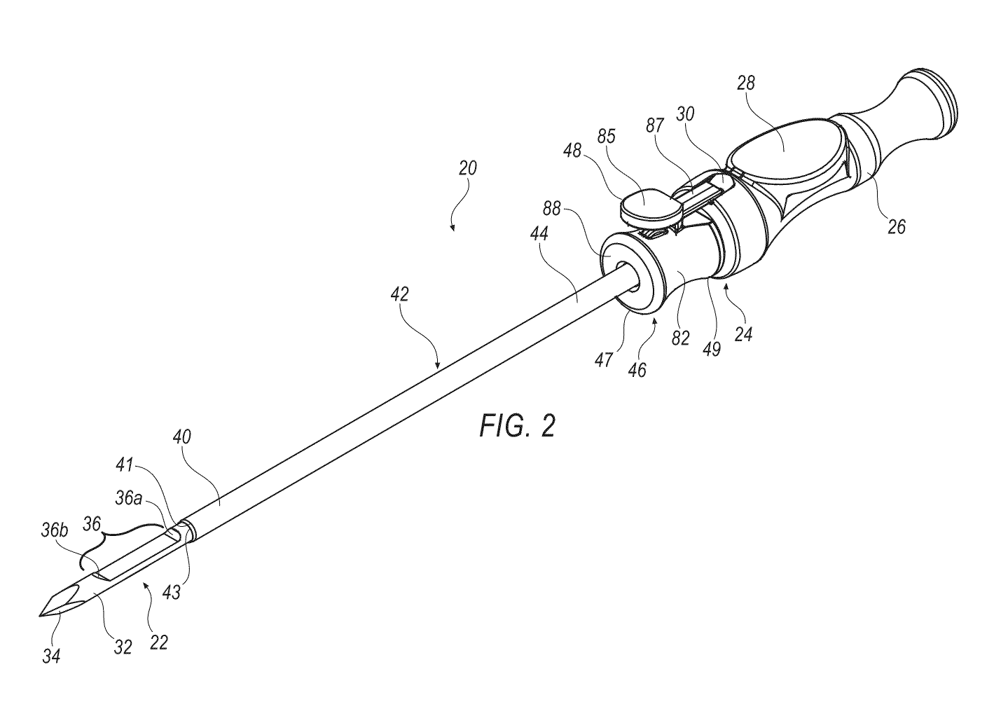

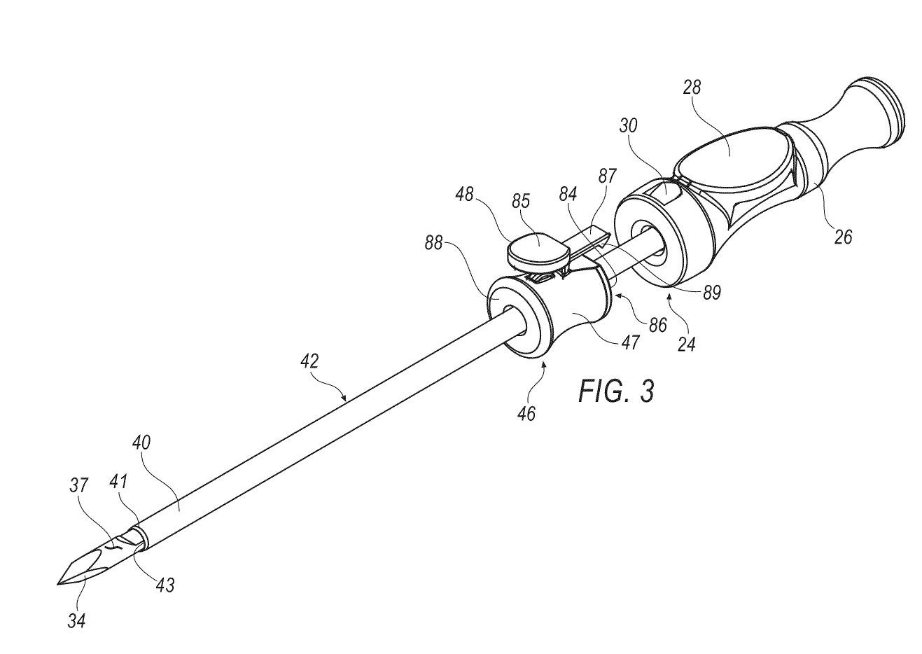

Turning now to FIGS. 2-3, an exemplary introducer assembly 20, which includes the introducer stylet body 22 received within and latched to an introducer cannula 42, is shown. In the illustration of FIGS. 2-3, introducer cannula 42 is configured so as to be generally cylindrical and is defined by a distal end 40 and a proximal end 44. Proximal end 44 further includes a latching hub 46. A lumen shaft extends between distal end 40 and proximal end 44.

As will be described in detail, introducer assembly 20 is particularly, but not necessarily, suited for use in biopsy procedures that identify a target biopsy site using various imaging modalities such as ultrasonography or magnetic resonance imaging (MRI). Indeed, in one exemplary arrangement, the imagable feature 36 is an echogenic feature, particularly suitable with ultrasonography. In another alternative arrangement, the imagable feature 36 is a radio-opaque feature particularly suitable with mammography. The assembly 20 may also be made of a MRI compatible, medical grade material, such as 316 stainless steel or Inconel™ 625. In one specific embodiment, the introducer stylet body 22 and the introducer cannula 42 are made of stainless steel.

In one exemplary configuration, latching hub 46 includes a generally annular hub portion 47 and a latch portion 48. The annular hub portion 47 includes a hub outer surface 82, a hub mating surface 84, (best seen in FIG. 3) a hub proximal end 86, and a hub distal end 88. The latch portion 48 includes a release button 85 and a latch 87 having a latch tab 89 (as seen best in FIG. 3). In one configuration, the latch 87 may extend generally parallel to the lumen shaft of the introducer cannula 42. In one embodiment, the latch tab 89 extends perpendicular to and toward the center line of the lumen shaft of the introducer cannula 42. The latch tab 89 cooperates with the latching tab interference portion 30 to secure the introducer cannula 42 to introducer stylet 22 and to prevent the introducer cannula 42 from rotating or laterally moving with respect to the introducer stylet 22 during use.

The latching hub 46 of the introducer cannula 42 defines an entrance opening 49 for receiving the introducer stylet 22 (and other medical devices).

In one configuration, the distal end 40 of the introducer cannula 42 may include a sharpened tapered edge 41 defining an exit opening 43. In one embodiment, (best seen in FIG. 2) sharpened tapered edge 41 starts at an inner diameter of the lumen and extends outwardly radially to an outer diameter surface to create the tapered edge 41. Tapered edge 41 provides for a smooth entry into the tissue area. The lumen shaft extends longitudinally starting at the proximal end 44 and terminating at the distal end 40 tapered edge 41. To maintain visibility of the imagable feature 36 when the introducer stylet 22 is received within the introducer cannula 42, the length of the introducer cannula 42, from the hub 46 to the tapered edge 41, does not exceed the length of introducer stylet shaft 21 from the handle 26 to a proximal edge 36a of the imagable feature 36.

In one embodiment, entry into the tissue area begins when the introducer cannula 42 is attached to the introducer stylet 22 with the latch hub 46. Because the introducer stylet 22 is positively latched to the latch hub 46 of the introducer cannula 42, the assembled assembly 20 provides for a predetermined length that permits more accurate location identification of treatment area. More specifically, as the imagable feature 36 is visible under one or more imaging modalities, once inserted, the operator will be able to visual verify the location of the imagable feature 36 prior to inserting a medical device into a patient.

After the introducer cannula 42 is positively attached to the introducer stylet 22, the operator grasps the handle 26 and presses the piercing tip 34 into the tissue. The piercing tip 34 enters the tissue and the operator applies further pressure sliding the assembly 20 into the tissue. The introducer cannula 42 enters skin and the operator applies further pressure while watching an imaging device as the assembly 20 creates an entry path to the targeted tissue area. The operator will complete the entry by applying continuous pressure to the assembly 20 until the imagable feature 36 reaches the target tissue. As stated above, in one specific configuration, the imagable feature 36 is sized to generally represent the size of an aperture of a biopsy device. Accordingly, the imagable feature 36 will provide visual confirmation of the location from which biopsy samples will be taken, i.e., the depth into which a distal end of a medical device will extend through the introducer cannula 42.

Once the operator verifies that the imagable feature 36 has reached the target area, the operator may release the introducer stylet 22 from the introducer cannula 42. Separation of the assembly 20 is accomplished when the operator holds the latching hub 46 and presses the release button 85, causing disengagement of the latch tab 89 from the latch tab interference portion 30. The operator then grasps the handle 26 and slides introducer stylet 22 out of the introducer cannula 42 and ultimately out of the tissue leaving the introducer cannula 42 within the patient's body. Thus, the introducer cannula 42 creates a direct pathway for the introduction of other medical devices, such as a biopsy device, for example. Upon completion of the medical procedure the operator will then slide the introducer cannula 42 and the medical device out of the body.

In some embodiments, the imagable feature 36 may be configured to also represent an orientation of an aperture of a medical device, such as a biopsy device. For example, as seen in FIGS. 1 and 2, the imagable feature 36 is configured with a generally planar surface, flanked by a proximal end 36a and a distal end 36b. In one particular embodiment, proximal end 36a and distal end 36b are disposed at an angle that tapers inwardly towards the generally planar surface from an outer surface 37 of the introducer stylet body 21. The generally planar surface is reflective, which will serve as an orientation indicator. More specifically, as the introducer stylet 22 is positioned in the targeted tissue, paying particular attention to the orientation indicator 28 as related to the imagable feature 36. Once the distal end 32 of the introducer stylet 22 reaches the targeted tissue, the operator will rotate the handle 26 to orient the imagable feature 36 in the direction of the sample to be taken. The orientation handle 28 will reflect the orientation of the imagable feature 36. Thus, once the orientation is set, and the introducer stylet 22 is removed, the orientation of the medical device will be set when the medical device is inserted into the introducer cannula 42 and locked thereto.

FIG. 3 is an illustration of the introducer assembly 20 with the latch hub 47 unlocked from the latch tab interference portion 30 of the introducer stylet 22. The illustration of FIG. 3 further demonstrates the interaction between the introducer cannula 42 and the introducer stylet 22. More specifically FIG. 3 demonstrates, as detailed above, the effect of releasing the latching hub 46 by pushing down on the release button 85, thereby releasing the latch 87 and the latch tab 89 so as to allow the introducer stylet 22 to be removed from the introducer cannula 42. Once released, the introducer cannula 42 may be left within the tissue with the proper orientation set for the addition of a core biopsy device, a syringe or other required device, as previously explained above.

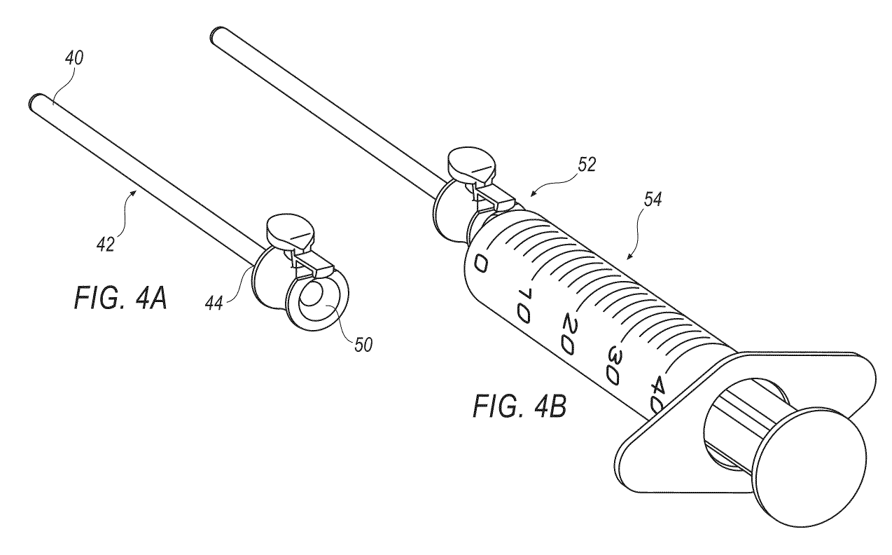

FIGS. 4A and 4B illustrate an alternative embodiment of the introducer cannula 42. In the illustration of FIG. 4A, introducer cannula 42 includes a hub 50 for sealingly connecting to a medical device. One such exemplary medical device is a syringe 54. In one embodiment, hub 50 may be configured with a luer type fitting that engages with a complimentary luer-type fitting positioned on a distal end 52 of syringe 54.

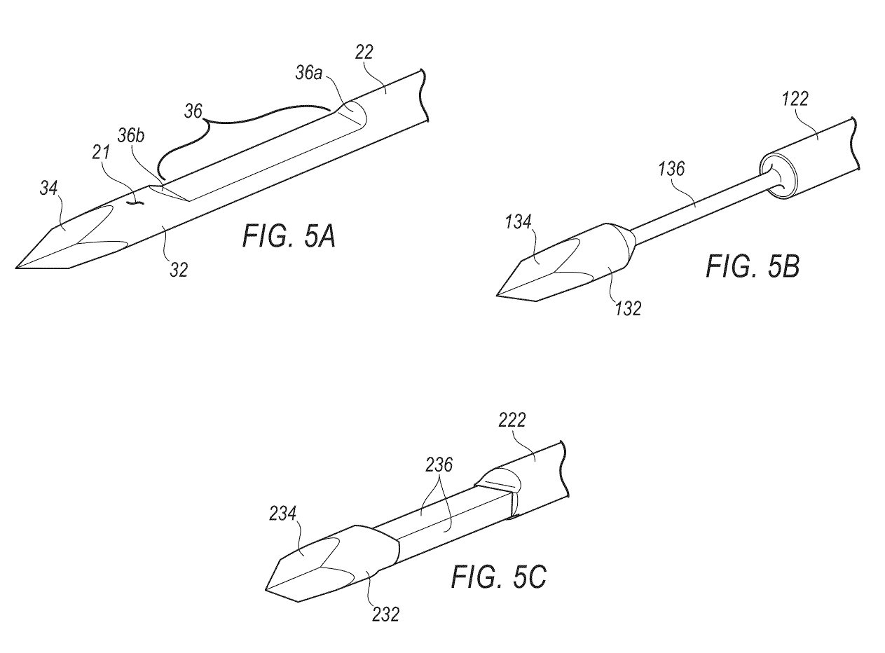

FIG. 5A illustrates further detail of the imagable feature 36, which is defined by the proximal end 36a and a distal end 36b. In one embodiment, the imagable feature 36 is a ground notch formed in a portion of the cylindrical introducer stylet 22. The imagable feature 36 is sized so as to represent an aperture size and shape of the biopsy device as referenced above in FIG. 1. The imagable feature 36, at a predetermined orientation and under a suitable imaging modality, creates a reflective surface as a result of the change in surface area or an applied surface treatment. This change creates a larger image reflection signature when using a suitable imaging device to guide the introducer stylet 22 into the target tissue area. Once the introducer stylet 22 is introduced into the target tissue area, the operator rotates the handle 26, setting the radial orientation of the ground notch of the imagable feature 36 on the introducer stylet 22 to the desired biopsy area. The radial orientation of the imagable feature 36 represents the aperture of the biopsy device relative to the target tissue once the biopsy device is disposed within the introducer cannula 42.

FIGS. 5B and 5C illustrate different embodiments of the imagable feature 36. As seen in FIG. 5B, an introducer stylet 122 having a turned diameter 136 is shown. The turned diameter 136 creates a signature visible from all orientations during imaging, though no aperture orientation will be visible in this particular embodiment. FIG. 5C includes an introducer stylet 222 having machined (and generally planar) surfaces 236, again creating a highly reflective signature visible from a specific orientation such that an aperture orientation would be visible.

The imagable features 36, 136 and 236 are all created using material removal techniques, including but not limited to, rough surface grinding with a carbon nitride grinding wheel, milling or turning the diameter on a lathe or other suitable material removal device. The imagable feature 36 may also be created through surface treatments such as but not limited to, grit blast, bead blast, laser abrasion, dimpling, or by applying an imagable coating or any other suitable method creating a surface capable of displaying a larger imagable reflective signature when using a suitable imaging device.

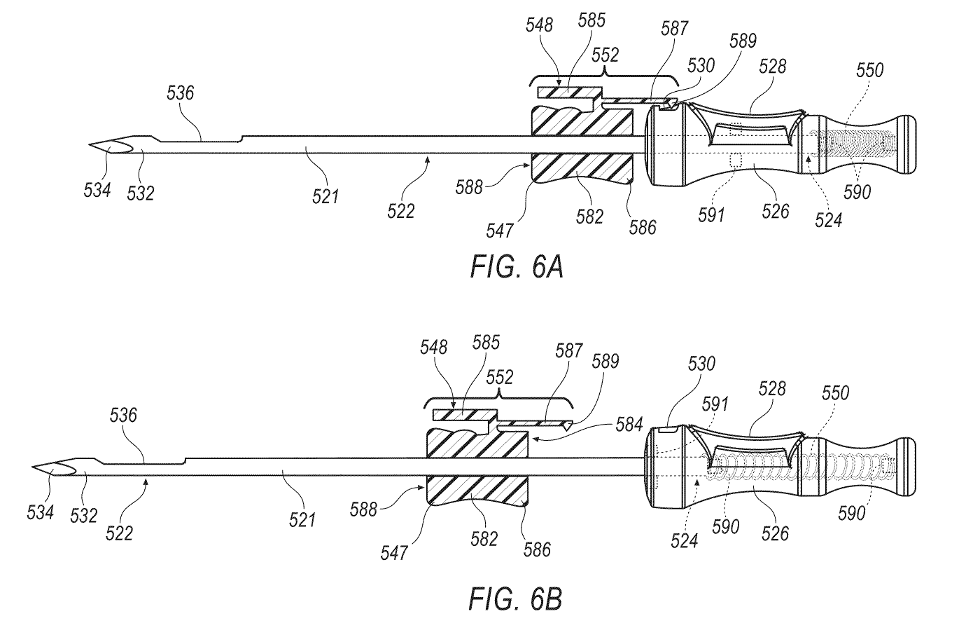

FIGS. 6A and 6B illustrate an exemplary spring loaded introducer stylet body 522. The introducer stylet body 522 is suited for use in an X-ray arrangement, as will be explained in further detail below. In the illustration of FIGS. 6A and 6B, introducer stylet body 522 is configured as generally cylindrical and defined by a distal end 532 and a proximal end 524. Distal end 532 includes a tissue piercing tip 534 sized for introduction into a patient's body. In one embodiment, piercing tip 534 is configured as a trocar tip. However, it will be appreciated that the piercing tip 534 may include other devices for piercing the patient's tissue, including without limitation, devices that use a laser or radio frequencies (RF) to pierce the tissue.

In the embodiment illustrated, introducer stylet body 522 extends from a handle 526. The introducer stylet body 522 may include an introducer stylet shaft 521 moveably received within the handle 526 and a firing mechanism 552 fixedly attached to a portion of the introducer stylet shaft 521. The handle 526 may include a latching tab interference portion 530 that operatively connects to the firing mechanism 552. The handle 526 may also include an imagable feature orientation indicator 528 so as to provide a visual orientation position of an imagable feature 536 on the distal end 532 of introducer stylet body 522 relative to the handle 526 (for those embodiments that include a generally planar reflective surface). In one exemplary arrangement, the imagable feature orientation indicator 528 may be configured as an ergonomic thumb pad. Alternatively, the imagable feature orientation indicator 528 may also be attained by printing a mark on the handle (not shown) that allows the operator to orient the imagable feature 536 to a target tissue area prior to the removal of the introducer stylet body 522 and the insertion of a core biopsy device within an introducer cannula 42 such as shown in FIGS. 2 and 3.

The firing mechanism 552 includes a generally annular hub portion 547 that carries latch portion 548. The annular hub portion 547 includes a hub outer surface 582, a hub mating surface 584, a hub proximal end 586, and a hub distal end 588. A first spring locator (shown in phantom as 590) is mounted on a proximal end 524 of the introducer stylet body 521. A second spring locator (also shown in phantom as 590) is mounted to an inside surface of the handle 526. A spring 550 is secured to the spring locators 590. A retaining collar 591 is fixedly secured to the introducer stylet body 521 and disposed with the handle 526 to prevent the proximal end 524 of the introducer stylet body 521 from disengaging from the handle 526. The latch portion 548 includes a release button 585 and a latch 587 having a latch tab 589 (as seen best in FIG. 3). The latch tab 589 may extend generally perpendicular to the introducer stylet shaft 521 of the introducer stylet body 522. In one embodiment, the latch 587 extends parallel to and toward the center line of the introducer stylet shaft 521 of the introducer stylet body 522. The annular hub portion 547 is mounted to the introducer stylet shaft 521 and travels outwardly from the handle 526 with the introducer stylet shaft 521 upon release of a spring 550.

The firing mechanism 552 prevents the introducer stylet body 522 from extending into a tissue area prematurely during a procedure. During a procedure, the introducer stylet 522 is mounted within a sterotactic cradle (not shown) having an upwardly extending wall portion (not shown), against which a proximal end of handle 526 is mounted. Once mounted, the introducer stylet 522 enters into the tissue area when the spring 550 is released from a pre-fire, compressed position (FIG. 6A) to an extended, fired position (FIG. 6B) and the introducer stylet shaft 521 is thrust into the tissue. The wall portion of the cradle will prevent the handle 526 from moving in the proximal direction when the spring 550 is released from the compressed position, thereby forcing the introducer stylet 521 to move in the distal direction and into the tissue.

Indeed, in operation, the operator grasps the handle 526, paying particular attention to the orientation indicator 528 as related to the imagable feature 536, and forces the piercing tip 534 into the tissue. The piercing tip 534 enters the tissue and the operator presses the release button 585, causing disengagement of the latch tab 589 from the latch tab interference portion 530. The force from the spring 550, releasing from a compressed position, (best seen in FIG. 6A) causes the introducer stylet shaft 521 to force itself through the tissue creating a path for the later introduction of other medical devices.

Once the operator has released the spring 550 to the fully extended position the operator will apply further pressure, while watching the imaging device, to guide the imagable feature 536 adjacent the targeted tissue. After the imagable feature 536 reaches the targeted area the operator will rotate the handle 526 to orient the imagable feature 536 in the direction of the sample to be taken. Upon proper orientation the operator may remove the introducer stylet 522 or leave the introducer stylet 522 in place while evaluating other procedures. To remove the introducer stylet 522 the operator grasps the handle 526 and slides the introducer stylet 522 out of the tissue leaving a path for the introduction of other medical devices, as stated above.

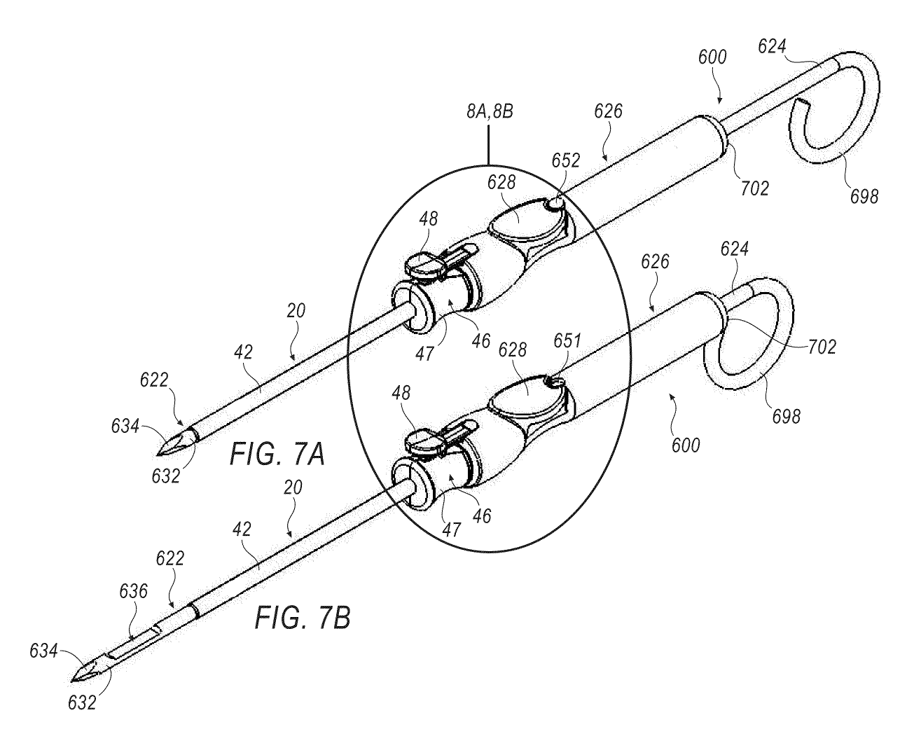

Referring now to FIGS. 7-9, an alternative embodiment of a spring loaded introducer stylet assembly 600 is shown. Referring first to FIGS. 7A and 7B, introducer stylet assembly 600 includes an introducer stylet body 622. In one exemplary arrangement, introducer stylet body 622 is configured as generally cylindrical and defined by a distal end 632 and a proximal end 624. Distal end 632 may include a tissue piercing tip 634 sized for introduction into a patient's body. It will be appreciated that the piercing tip 634 may include other devices for piercing the patient's tissue, including, without limitation, devices that use a laser or radio frequencies (RF) to pierce the tissue. An imagable feature 636 may be positioned adjacent to distal end 632 of introducer stylet 622. While shown as a generally planar surface, it is appreciated that imagable feature 636 may be formed in a variety of configurations, including, but not limited to, those configurations shown and described above in connection with FIGS. 5A-5C.

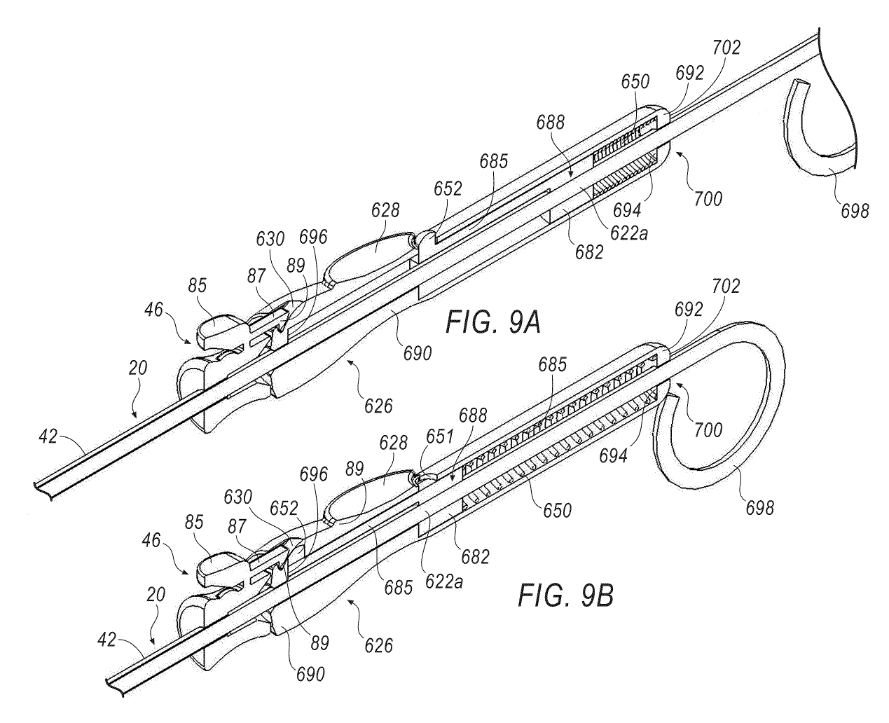

In the illustrated arrangement, introducer stylet body 622 is positioned in sliding arrangement through a handle 626. A firing button 652 (to be explained in further detail below) is provided that extends through an opening 651 (best seen in FIG. 9B) in handle 626 when introducer stylet assembly 600 is in a “pre-fire” configuration (as shown in FIGS. 7A, 8A, and 9A). In one exemplary arrangement, firing button 652 is positioned so as to be adjacent to an imagable feature orientation indicator 628. As with the other embodiments disclosed herein, imagable feature orientation indicator 628 may be configured as an ergonomic thumb pad. Alternatively, the imagable feature orientation indicator 628 may also be configured by printing a mark on handle 626 (not shown) that allows the operator to selectively orient the imagable feature 636 to a target tissue area prior to the removal of the introducer stylet body 622 and the insertion of a biopsy device.

Introducer stylet assembly 600 may also be used with an introducer assembly 20 (described in greater detail in connection with FIGS. 2-3). As may be seen, introducer stylet assembly 20 includes an introducer cannula 42 and a latching hub 46. Latching hub 46 includes a generally annular hub portion 47 and a latch portion 48. The latch portion 48 includes a release button 85 and a latch 87 having a latch tab 89 (as seen best in FIGS. 9A-9B). The latch tab 89 cooperates with a latching tab interference portion 630 formed on a surface of handle 626 to prevent the introducer cannula 42 from rotating or laterally moving with respect to the introducer stylet body 622 during use.

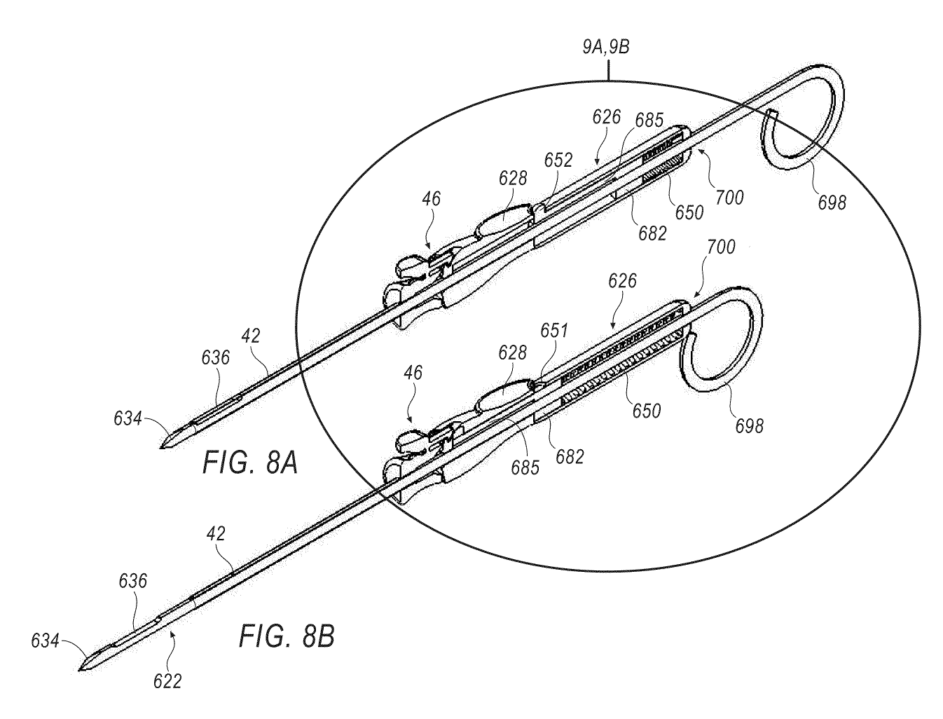

As best shown in FIGS. 8 and 9, introducer stylet assembly 600 further includes a slidable carriage 688 and a biasing member 650. Slidable carriage 688 has a body member 682 and an arm member 685. Arm member 685 extends outwardly from body member 682 and carries firing button 652.

Slidable carriage 688 is fixedly secured to a portion of introducer stylet body 622a and disposed within a chamber 690 formed within handle 626. Biasing member 650 is also positioned within chamber 690, adjacent a proximal end 692 of handle 626. Biasing member 650 is positioned between body member 682 of carriage 688 and an internal surface 694 of chamber 690.

In operation, when introducer stylet assembly 600 is in the “pre-fire” configuration (FIGS. 8A and 9A), carriage 688 is retracted and held in the pre-fire configuration by firing button 652 that extends upwardly through opening 651. Body member 682 serves as a stop member for biasing member 650 (which may be a spring), such that when introducer stylet assembly 600 is in the “pre-fire” configuration, biasing member 650 is compressed between body member 682 and internal surface 694 of chamber 690.

To move introducer stylet assembly 600 into a “fired” configuration (see FIGS. 8B and 9B), firing button 652, which is selectively compressible, is depressed thorough opening 651. Once cleared from opening 651, biasing member 650 acts against body member, thereby “firing” carriage 688, which is carrying introducer stylet body 622, forward. The firing button 652 cooperates with a forward wall member 696 to limit the forward extent that introducer stylet body 622 may fire.

Further, proximal end 624 of introducer stylet body 622 may also be configured with a stop member 698 that serves to prevent introducer stylet body 622 from exiting handle 626 during the firing process. For example, as may be seen in FIGS. 8-9, proximal end 624 of introducer stylet body extends outwardly from an opening 700. In one exemplary arrangement, a stop member 698 is attached to proximal end 624. When introducer stylet assembly 600 is positioned within the fired configuration (7A, 8A, and 9A), a portion of stop member 698 contacts an outside surface 702 of proximal end 692 of handle 626. Stop member 698 may be configured integrally with introducer stylet body 622 or a separate piece.

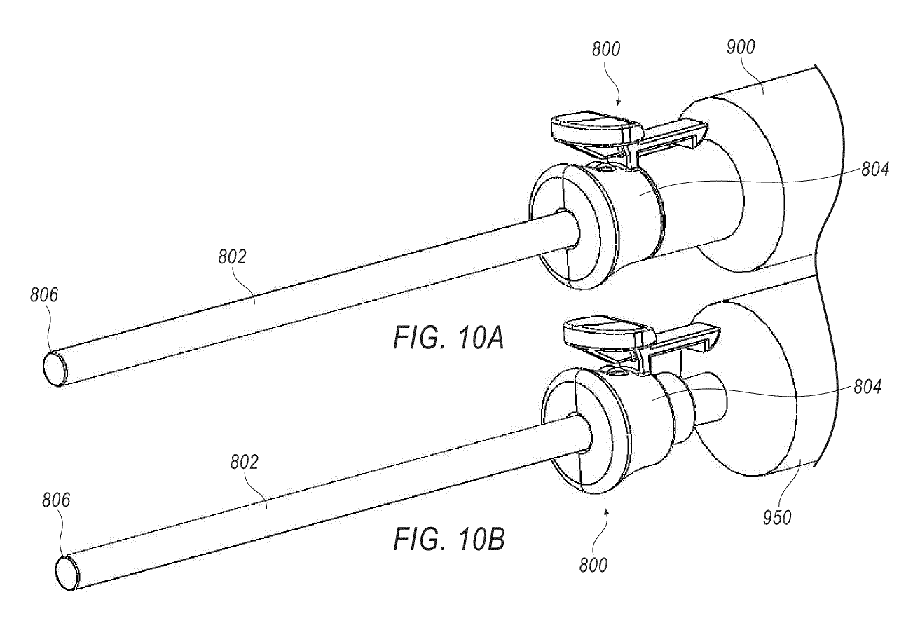

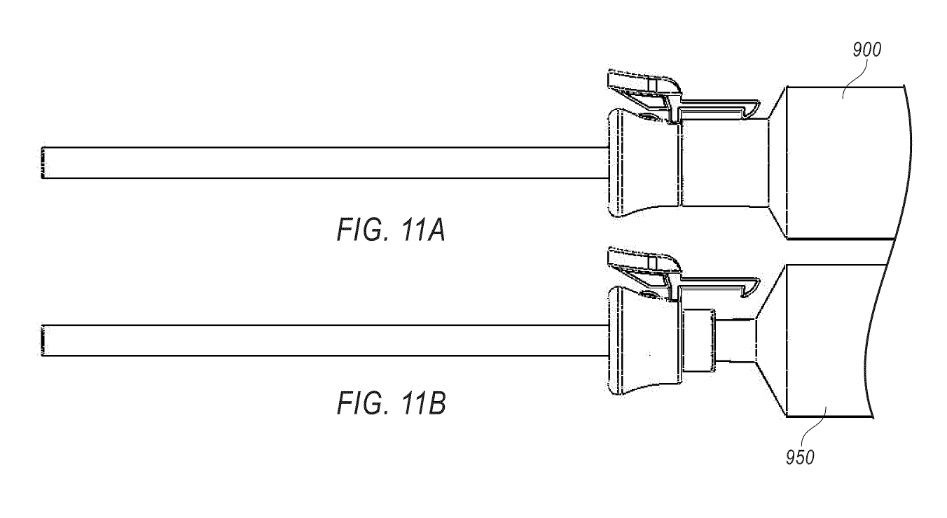

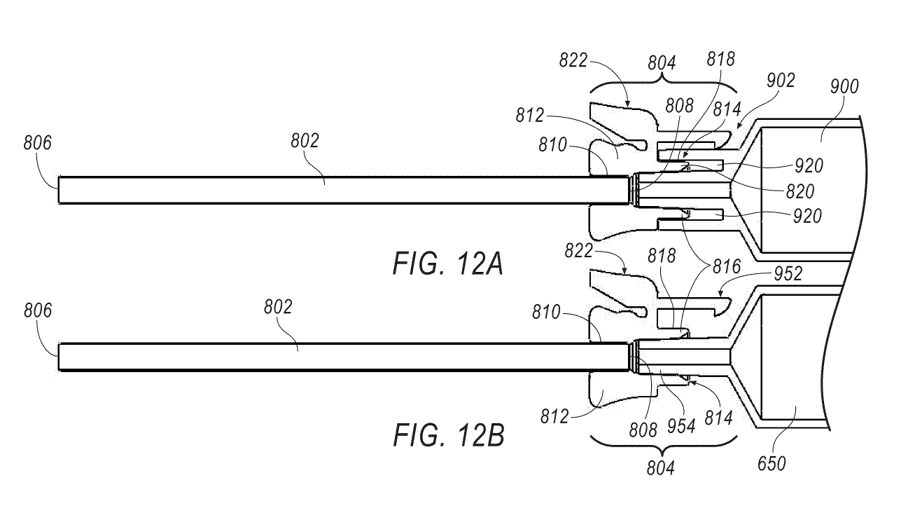

Referring now to FIGS. 10-12, an alternative embodiment of an introducer cannula assembly 800 is shown. Introducer cannula assembly 800 comprises an introducer cannula 802 and an introducer hub 804. Introducer cannula 802 is configured so as to be generally cylindrical and is defined by a distal end 806 and a proximal end 808 (best seen in FIGS. 12A and 12B. Proximal end 808 of introducer cannula 802 is fixedly secured within a channel 810 formed in a distal portion 812 of introducer hub 804.

Introducer hub 804 further includes a proximal portion 814. Proximal portion 814 further includes a gripping member 816 that is disposed around a mounting groove 818. In one exemplary embodiment, gripping member 816 is configured as an annular member that extends around mounting groove 818. In another embodiment, gripping member 816 may be configured as a series of projections positioned around mounting groove 818. Gripping member 816 is constructed of a compressible material, as will be explained in further detail below.

Introducer hub 804 is designed to allow selective fixed engagement with a variety of medical devices. More specifically, gripping member 816 is sized to engage both luer lock type syringes 900, as well as luer taper type syringes 950. However, unlike traditional fittings for introducers that are threaded or require a twist-type engagement (such as bayonet fittings), gripping member 816 is designed to fit multiple types of medical devices with a quick interference-type fit.

For example, referring to FIG. 12A, introducer hub 804 gripping members 816 may be engaged with an internal mounting groove 920 formed in a distal end 902 of luer lock type syringe 900 in an interference fit. To facilitate such engagement, an end portion 820 of gripping member 818 may be configured with a taper. To effect engagement between introducer hub 804, end portion 820 of gripping member 816 is press fit into internal mounting groove 920. The compressible material is compressed within mounting groove 920, thereby providing an interference fit between introducer hub 804 and mounting groove 920, without requiring any twisting motion.

Referring to FIG. 12B, introducer hub 804 gripping members 816 may be engaged with an external surface 952 of luer taper type syringe 950. More specifically, a distal end 954 of syringe 950 is received within mounting groove 818 of introducer hub 804. The compressible material expands around distal end 954 and gripping members 816 engage external surface 952 in an interference fit without requiring any twisting motion.

In addition to gripping members 816 and mounting groove 818, introducer hub 804 may also be optionally provided with a latch portion 822, similar to those described above in connection with FIGS. 1-3. In such an embodiment, introducer hub 804 may also selectively engage those devices that include a latch interference portion.

While the present disclosure has been particularly shown and described with reference to the foregoing preferred embodiments, it should be understood by those skilled in the art that various alternatives to the embodiments of the disclosure described herein may be employed in practicing the disclosure without departing from the spirit and scope of the disclosure as defined in the following claims. It is intended that the following claims define the scope of the disclosure embodiments within the scope of these claims and their equivalents be covered thereby. This description of the disclosure should be understood to include all novel and non-obvious combinations of elements described herein, and claims may be presented in this or a later application to any novel and non-obvious combination of these elements. The foregoing embodiment is illustrative, and no single characteristic or element is essential to all possible combinations that may be claimed in this or a later application.

What is claimed is:

1. An introducer assembly, comprising:
an introducer cannula defined by a distal end and a proximal end and having a lumen extending therebetween;
an introducer hub operatively connected to the introducer cannula, wherein the introducer hub is configured for selective connection with multiple medical devices; and
a introducer stylet body defined by a proximal end and a distal end, wherein the introducer stylet body is removably disposed within the introducer cannula.

2. The introducer assembly of claim 1, wherein the introducer stylet body is defined by a proximal end and a distal end, wherein the distal end of the introducer stylet body further includes an imagable feature having a predetermined size.

3. The introducer assembly of claim 1, further comprising a biasing member operatively connected to a portion of the introducer stylet body.

4. The introducer assembly of claim 3, further comprising a slidable carriage that is fixedly connected to a portion of the introducer stylet body, wherein the slidable carriage further comprises a firing button thereon.

5. The introducer assembly of claim 4, wherein the slidable carriage further comprises a body member and an arm member extending from the body member.

6. The introducer assembly of claim 5, wherein the firing button is disposed on the arm member.

7. The introducer assembly of claim 4, wherein the firing button is selectively compressible.

8. The introducer assembly of claim 3, wherein the biasing member and slidable carriage is positioned within a housing, and wherein a proximal end of the introducer stylet body is disposed proximally of the housing.

9. The introducer assembly of claim 8, wherein the biasing member is positioned between a proximal end of the slidable carriage and an internal wall of the housing.

10. The introducer assembly of claim 2, further comprising a handle operatively connected to the introducer stylet body, wherein the introducer stylet body further comprises an orientation indicator.

11. The introducer assembly of claim 3 further comprising a biasing member operative connected to a proximal end of the introducer stylet body, wherein one end of the biasing member is mounted on a locator disposed on the proximal end of the introducer stylet body.

12. The introducer assembly of claim 11 further comprising a firing mechanism fixedly connected to the introducer stylet body, the firing mechanism including a latch tab that is operatively connected to a release button.

13. The introducer assembly of claim 1, wherein the introducer stylet further comprises a latch tab interference portion, wherein the latch tab interference portion may be operatively engaged with a portion of the introducer hub so as to secure the introducer stylet within the introducer cannula and prevent the introducer stylet and introducer cannula from moving independently from one another when positioned in a locked position.

14. The introducer assembly of claim 13, wherein the introducer hub further comprises a latch tab operative connected to a release.

15. The introducer assembly of claim 1, wherein the introducer hub further comprises a channel disposed in a distal portion thereof, wherein the channel receives a proximal end of the introducer cannula therein.

16. The introducer assembly of claim 15, wherein introducer hub further comprises a proximal portion that includes at least one gripping member disposed around a mounting groove, wherein the gripping member is configured to engage with a portion of a medical device in an interference fit.

17. The introducer assembly of claim 16, wherein the gripping member is constructed as an annular member.

18. The introducer assembly of claim 16, wherein the introducer hub further comprises a latch portion that includes a latch member and a release member.

19. The introducer assembly of claim 1, wherein the introducer hub is constructed of a compressible material.

20. An introducer assembly, comprising:
an introducer cannula defined by a distal end and a proximal end and having a lumen extending therebetween;
an introducer hub, wherein the introducer hub further comprises a channel disposed in a distal portion thereof, wherein the channel receives a proximal end of the introducer cannula therein; wherein the introducer hub also comprises a proximal portion that includes at least one gripping member disposed around a mounting groove, wherein the gripping member is configured to engage with a portion of a medical device in an interference fit; and wherein the introducer hub also comprises a latch portion that includes a latch member and a release member that is configured for selective connection with a latch tab interference portion disposed on a medical device; and
an introducer stylet body defined by a proximal end and a distal end removably disposed within the introducer cannula, wherein the distal end of the introducer stylet body includes an imagable feature having a predetermined size and the proximal end is connected to a handle member having an orientation indicator.
